(19) United States
(12) Patent Application Publication
(10) Pub. No.: US 2025/0177339 A1
(43) Pub. Date: Jun. 5, 2025

(54) Methods for Treating Tauopathy

(71) Applicant: BioJiva LLC, San Jose, CA (US)

(72) Inventor: Mikhail Sergeevich SHCHEPINOV, Oxford (GB)

(73) Assignee: BioJiva LLC, San Jose, CA (US)

(21) Appl. No.: 18/767,538

(22) Filed: Jul. 9, 2024

Related U.S. Application Data
(63) Continuation of application No. 17/175,397, filed on Feb. 12, 2021, now abandoned.
(60) Provisional application No. 62/976,958, filed on Feb. 14, 2020.

(51) Int. Cl. A61K 31/202; A61K 31/122; A61K 31/201; A61K 31/231; A61K 31/232; A61K 31/355; A61K 31/375; A61P 25/28

(52) U.S. Cl. CPC A61K 31/202; A61K 31/122; A61K 31/201; A61K 31/231; A61K 31/232; A61K 31/355; A61K 31/375; A61P 25/28

(57) ABSTRACT

Disclosed are uses of isotopically modified polyunsaturated compounds for treating, ameliorating or inhibiting the progression of a neurodegenerative disease or condition related to tauopathy in a subject in need thereof. In certain embodiments, the isotopically modified polyunsaturated compounds are deuterated polyunsaturated fatty acids or derivatives thereof.

### CROSS-REFERENCE TO RELATED APPLICATIONS

This application claims the benefit of U.S. Provisional Patent Application Ser. No. 62/976,958 filed on Feb. 14, 2020 under the provisions of 35 U.S.C. § 119 (e)(1) which application is incorporated by reference herein in its entirety.

### BACKGROUND OF THE INVENTION

### Field of the Invention

This disclosure relates to methods and compositions for treating tauopathy. In some embodiments, these methods and compositions relate to use of deuterated polyunsaturated fatty acids or derivatives thereof in treating diseases mediated by tauopathy.

### Description of the Related Art

Tauopathy is a subgroup of Lewy body diseases or proteinopathies. Tauopathy comprises several neurodegenerative conditions involving the aggregation of tau protein into insoluble tangles, with aggregates forming from hyperphosphorylation of tau protein in the human brain. These neurodegenerative conditions fall under a broader category of Lewy body diseases or proteinopathies. Specific conditions related to tauopathy include, but are not limited to, argyrophilic grain disease (AGD), chronic traumatic encephalopathy (CTE), corticobasal degeneration (CBD), frontotemporal dementia and parkinsonism linked to chromosome 17 (FTDP-17), ganglioglioma, gangliocytoma, lipofuscinosis, lytico-bodig disease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS), and subacute sclerosing panencephalitis (SSPE). Wang et al., Nature Rev. Neurosci. 2016; 17:5 and Arendt et al., Brain Res. Bulletin 2016; 126:238. Tauopathies often overlap with synucleinopathies.

Steele-Richardson-Olszewski syndrome or progressive supranuclear palsy (PSP) is a neurodegenerative disease involving the gradual deterioration and death of specific volumes of the brain. The condition leads to symptoms including loss of balance, slowing of movement, difficulty moving the eyes, and dementia. A variant in the gene for tau protein called the H1 haplotype, located on chromosome 17, has been linked to PSP. Besides tauopathy, mitochondrial dysfunction seems to be a factor involved in PSP. Especially, mitochondrial complex I inhibitors are implicated in PSP-like brain injuries.

It is well understood that oxidative stress plays the major role in neurodegenerative tauopathies. Ganguly et al., Drug Design, Dev. And Therapy 2017; 11:797. More specifically, lipid peroxidation (LPO) is recognized as a particularly relevant tauopathy-inducing factor. Porter N A, Methods Enzymol. 1(984) 105:273; Gomez-Ramos, et al., J. Neurosci. Res., (2003) 71:863; and Liu, et al., Free Rad. Biol. Med., (2005) 38:746.

Mitochondrial dysfunction, another common denominator of various proteinopathic neurological diseases is closely linked to oxidative stress and LPO. It is well known that such a dysfunction comprises various defects including mitochondrial fission and fusion, mitophagy, autophagy, apoptosis, signaling, calcium homeostasis and OxPhos pathway malfunction, all of which are directly linked to neurodegeneration. Murphy, et al., J. Cereb. Blood Flow Metab., (1999) 19:231-245; Lin, et al., Nature (2006) 443:787-795. These and other parameters such as mitochondrial shape and membrane curvature are directly influenced by the lipid bilayer environment and lipid metabolism. Aufschnaiter, et al., Cell Tissue Res., (2017) 367:125-140. Mitochondrial lipid membranes are rich in polyunsaturated fatty acids (PUFA) which are particularly prone to the ROS initiated oxidation. Importantly, oxidation of PUFAs proceeds through the chain reaction format, whereby one initiating ROS-driven damage event generates substantial damage and multiple toxic products. These, in turn, give rise to more ROS sustaining the vicious circle. Accumulation of reactive oxygen species (ROS) and LPO are thought to play pivotal roles in the pathophysiology of Steele-Richardson-Olszewski syndrome (SROS). Odetti, et al., J. Neuropathol. Exp. Neurol., (2000) 59:393-397; and Zarkovic, Molec. Aspects Med., (2003) 24:293-303. There remains a need to develop new pharmaceutical treatments for neurodegenerative tauopathies.

It has now been discovered that isotopically modified polyunsaturated fatty acids, estersand derivatives thereof are useful for treating, mitigating, or inhibiting the progression of a neurodegenerative disease or condition related to tauopathy.

### SUMMARY

In one aspect, this disclosure relates to a method of treating or ameliorating a neurodegenerative diseases or conditions that are mediated at least in part by tauopathy; or inhibiting the progression of a neurodegenerative disease or condition related to tauopathy in a patient in need thereof, comprising administering a first effective amount of one or more deuterated polyunsaturated lipids or pharmaceutically acceptable salts thereof to the subject during a first period of time. In some embodiments, the method further comprises administering a second effective amount of one or more deuterated polyunsaturated lipids or pharmaceutically acceptable salts thereof to the subject during a second period of time. In some embodiments, the neurodegenerative disease or condition related to tauopathy is selected from the group consisting of argyrophilic grain disease (AGD), chronic traumatic encephalopathy (CTE), corticobasal degeneration (CBD), frontotemporal dementia and parkinsonism linked to chromosome 17 (FTDP-17), ganglioglioma, gangliocytoma, lipofuscinosis, lytico-bodig disease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS), and subacute sclerosing panencephalitis (SSPE). In some further embodiments, the neurodegenerative disease or condition is not Alzheimer's disease, Parkinson's disease, or frontotemporal dementia.

The pathology of each of these diseases involves the aggregation of tau protein into insoluble tangles, with aggregates forming from hyperphosphorylation of tau protein in the human brain.

In one embodiment, this disclosure provides for a method for reducing lipid peroxidation in neurons wherein said lipid peroxidation is associated with abnormal tubulin associated units (tau) characteristic of tauopathy, said method comprises:
contacting said neurons exhibiting tauopathy with a sufficient amount of a deuterated polyunsaturated fatty acids over a period of time sufficient to accumulate such fatty acids in the neurons and, in particular, in the neuronal membrane,
wherein said deuterated polyunsaturated fatty acids incorporated into said neurons attenuate lipid peroxidation in said neurons thereby stabilizing the neurons against neuronal death associated with abnormal tau.

In one embodiment, this disclosure provides for a method for reducing lipid peroxidation in neurons wherein said lipid peroxidation is associated with abnormal tubulin associated units (tau) in the brain characteristic of tauopathy, said method comprises: administering to a patient at risk of or suffering from tauopathy a sufficient amount of a deuterated polyunsaturated fatty acids over a period of time sufficient to accumulate such fatty acids in the neurons and, in particular, in the neuronal membrane of said patient, wherein said deuterated polyunsaturated fatty acids incorporated into said neurons attenuate lipid peroxidation in said neurons thereby stabilizing the neurons against neuronal death associated with abnormal tau.

In some embodiments, there are provided methods for reducing lipid peroxidation in neurons, wherein said lipid peroxidation is associated with abnormal tubulin associated units (tau) characteristic of tauopathy, said method includes contacting said neurons with a sufficient amount of a deuterated polyunsaturated fatty acid (PUFA) or ester or derivative thereof, over a period of time sufficient to accumulate said deuterated PUFA or ester or derivative thereof, in said neurons; wherein said deuterated PUFA or ester or derivative thereof accumulated in said neurons stabilizes the neurons against neuronal death associated with abnormal tau.

In some embodiments, there are provided methods for reducing lipid peroxidation in neurons of a patient, wherein said lipid peroxidation is associated with abnormal tubulin associated units (tau) characteristic of tauopathy, said method includes administering to the patient a sufficient amount of a deuterated polyunsaturated fatty acid (PUFA) or ester or derivative thereof, over a period of time sufficient to accumulate said deuterated PUFA or ester or derivative thereof in the neurons inclusive of the neuronal membrane of said patient, wherein said accumulated, deuterated PUFA or ester or derivative thereof attenuates lipid peroxidation in said neurons thereby stabilizing the neurons against neuronal death associated with abnormal tau.

In some embodiments, there are provided methods of treating, ameliorating, or inhibiting the progression of a neurodegenerative disease or condition related to tauopathy in a subject, the method includes administering a first effective amount of one or more deuterated polyunsaturated lipids or pharmaceutically acceptable salts thereof to the subject during a first period of time.

In some embodiments, the neurodegenerative disease or condition related to tauopathy is selected from the group consisting of argyrophilic grain disease (AGD), chronic traumatic encephalopathy (CTE), corticobasal degeneration (CBD), frontotemporal dementia and parkinsonism, ganglioglioma, gangliocytoma, lipofuscinosis, lytico-bodigdisease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS) (also referred to as progressive supranuclear palsy—PSP), subacute sclerosing panencephalitis (SSPE), Alzheimer's disease, or Lytico-bodig disease.

In some embodiments, the neurodegenerative disease or condition is suspected SROS.

In some embodiments, the deuterated PUFA or ester or derivative there of is selected from the group consisting of a deuterated fatty acid, a deuterated fatty acid ester, a deuterated fatty acid thioester, a deuterated fatty acid amide, a fatty acid deuterated phosphate ester, or a phospholipid derivative, and wherein at least one or more of the bis-allylic position position of the deuterated PUFA or ester or derivative thereof is a site of deuterium substitution.

In some embodiments, the compositions may further include deuterium substitution at at least one further allylic site.

In some embodiments, the polyunsaturated lipid has a structure of Formula (I):

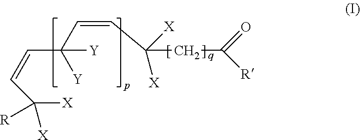

wherein:
R is hydrogen or optionally substituted C₁-C₁₀ alkyl, wherein said optional substitution is at least one deuterium;
R′ is —OR¹, —SR², —O(CH₂)CH(OR³)CH₂(OR⁴), —NR⁵R⁶

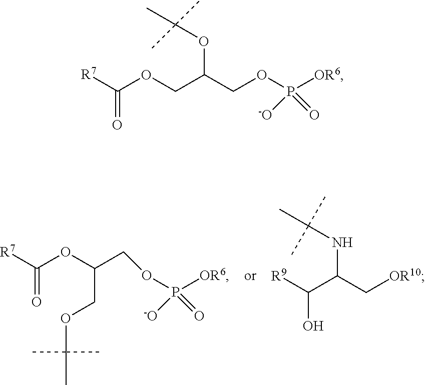

R¹ and R² is H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl;
each of R₃ and R₄ is independently H, optionally substituted —C(═O)C₁-C₂₁ alkyl, optionally substituted —C(═O)C₂-C₂₁ alkenyl, or optionally substituted —C(═O)C₂-C₂₁ alkynyl;
each of R⁵ and R⁶ is independently H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl; or R⁵ and R⁶ together with the nitrogen atom to which they are attached form an optionally substituted 3 to 10 membered heterocyclyl;
R⁷ is optionally substituted C₁-C₂₁ alkyl, optionally substituted C₂-C₂₁ alkenyl, or optionally substituted C₂-C₂₁ alkynyl;
R⁹ is optionally substituted C₈-C₂₁ alkyl, optionally substituted C₈-C₂₁ alkenyl, or optionally substituted C₈-C₂₁ alkynyl;
R¹⁰ is H,

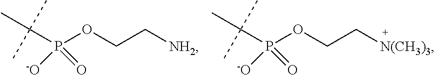

a mono-saccharide, a di-saccharide, or an oligosaccharide;
each X and Y is independently H or D, provided that at least one of X and optionally, one or more Y is D; and
each of p and q is independently an integer of 1, 2, 3, 4, 5, 6, 7, 8, 9 or 10;
or a mixture thereof.

In some embodiments, each of Y is D.

In some embodiments, R is methyl, C₄ alkyl, or C₇ alkyl, each optionally substituted with one or more D.

In some embodiments, the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

In some embodiments, the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

In some embodiments, the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

In some embodiments, ester OR¹ is an alkyl ester, a triglyceride, a diglyceride, or a monoglyceride.

In some embodiments, R¹ is ethyl.

In some embodiments, the deuterated PUFA or ester or derivative thereof is selected from 11,11-D2-linoleic acid, 11,11,14,14-D4-linolenic acid, 13,13-D2-arachidonic acid, 7,7,10,10,13,13-D6-arachadonic acid, 7,7,10,10,13,13,16,16-D8-eicosapentaenoic acid, or 6,6,9,9,12,12,15,15,18,18-D10-docosahexaenoic acid, or ethyl esters thereof.

In some embodiments, the mixture of deuterated polyunsaturated lipids has a deuteration degree of at least 50% at the bis-allylic positions.

In some embodiments, the mixture of deuterated polyunsaturated lipids has a deuteration degree of at least 70% at the bis-allylic positions.

In some embodiments, the one or more deuterated PUFA or ester or derivative thereof are co-administered with at least one antioxidant.

In some embodiments, the one or more deuterated PUFA or ester or derivative thereof are co-administered with at least one antioxidant.

In some embodiments, the one or more deuterated PUFA or ester or derivative thereof are co-administered with at least one antioxidant.

In some embodiments, the antioxidant comprises Coenzyme Q, idebenone, mitoquinone, mitoquinol, vitamin C, or vitamin E, or combinations thereof.

In some embodiments, frontotemporal dementia and parkinsonism is linked to chromosome 17 (FTDP 17).

### DETAILED DESCRIPTION

Embodiments of this disclosure relate to the method and use of isotopically modified polyunsaturated lipids such as deuterated polyunsaturated fatty acids and derivatives thereof for treating or ameliorating a disease or condition related to a neurodegenerative disease or condition related to tauopathy. In some embodiments, such tauopathy related neurodegenerative disease or condition is not Alzheimer's disease, Parkinson's disease, or frontotemporal dementia. In some embodiments, such tauopathy related neurodegenerative disease is Steele-Richardson-Olszewski syndrome (SROS) or progressive supranuclear palsy (PSP).

In some instances, the method or use described herein also reduces isoprostane interaction with phosphorylate-tau protein so that the latter can be dephosphorylated and cleared. As a result, it reduces or mitigates the toxicity caused by tau accumulation.

In some instance, the method or use described herein also prevents the cross linking of tau and phosphor-tau in the mid-brain in SROS subjects. In some cases, the method or use also reverses SROS by preventing mitochondrial cell death of neurons.

In some instance, the method or use described herein reverses SROS by synergistically preventing mitochondrial cell death of neurons and blocking down-stream toxic effects of isoprostane-induced failure to clear phosphorylated tau.

However, prior to providing a more detailed description, the following terms will first be defined.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting.

The use of the term “having” as well as other forms, such as “have”, “has,” and “had,” is not limiting.

As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

The term “about” as used herein, refers to a quantity, value, number, percentage, amount, or weight that varies from the reference quantity, value, number, percentage, amount, or weight by a variance considered acceptable by one of ordinary skill in the art for that type of quantity, value, number, percentage, amount, or weight. In various embodiments, the term “about” refers to a variance of 20%, 15%, 10%, 9%, 8%, 7%, 6%, 5%, 4%, 3%, 2% or 1% relative to the reference quantity, value, number, percentage, amount, or weight.

As used herein, the term “tauopathy” in the singular and “tauopathies” in the plural refer(s) to well-recognized set of clinically, biochemically, and morphologically heterogenous neurodegenerative diseases where the pathology of such diseases includes both soluble prefibrillar aggregates of tau (tubulin associated unit) proteins that cause the most damage to neurons and the deposition of abnormal tau in the brain (collectively “abnormal tau”). Many recognized neurodegenerative diseases are characterized as such independent of whether there is deposition of tau. Hence, many of these diseases have patients with or without tauopathy. However, as defined herein, only those patients having abnormal tau are included as a subset of a recognized disease. Abnormal tau is a rogue protein resulting in oxidative damage to neurons and, in particular to the fatty acids in the neuronal membrane, leading to neuronal death.

The following summary by Esteras et al. (“Mitochondrial Calcium Deregulation in the Mechanism of Beta-Amyloid and Tau Pathology,” Cell, 9:9 2135 (2020).) provides a detailed summary of tauopathy. “Tau protein (tubulin-associated unit) refers to microtubule-associated proteins. It is a soluble, natively unfolded, and phosphorylated protein, ubiquitously expressed in most tissues and organs. This protein exists as six alternatively spliced isoforms and is encoded by a single gene, mapt, that is located on chromosome 17 in humans. Tau is found in all cellular and subcellular compartments but is most prominent in the axons of neurons of the central nervous system. Tau protein plays an important role in neuronal physiology, in microtubule assembly and dynamics, in promoting axonal outgrowth, axonal transport and in signal transduction. Physiological and pathological activity of tau is dependent on the phosphorylation (tau is phosphoprotein) and alternative splicing and on the level of aggregation. The soluble prefibrillar aggregates of tau proteins cause the most damage to neurons. In disease, tau dissociates from microtubules and forms large, primarily intracellular, β-sheet rich fibrils. Tau protein is involved in the pathogenesis of many neurodegenerative diseases, specifically in Alzheimer's disease and frontotemporal dementia. Pathologies and dementias of the nervous system are associated with tau proteins that have become defective and no longer stabilize microtubules properly. The abnormal tau function leads to the deficits in fast axonal transport, dystrophic neurites, and abnormal mitochondrial distribution. This abnormal distribution of mitochondria is more likely to be induced by impairment the fission and fusion of mitochondria by tau. It also has been shown that in human tau transgenic mice and flies, F-actin is increased, which disrupts the physical association of mitochondria and the fission protein DRP1, leading to mitochondrial elongation. The resulting neurotoxicity can be rescued either by reducing mitochondrial fusion, or by enhancing fission, or by reversing actin stabilization. The possible effect of tau on mitochondrial complex I have been shown triple knockout Alzheimer's disease mouse mitochondria. The 10+16 intronic mutation in MAPT gene, encoding tau increase in the production of 4R tau isoforms, which are more prone to aggregation. Human iPSC derived neurons with this mutation are associated with partially suppressed complex Idriven respiration that leads to F1Fo-ATPase to be switched in reverse mode. This combination increased mitochondrial membrane potential that trigger ROS production in electron transport chain which causes oxidative stress and cell death.” (citations and Figure references omitted).

Clinically, biochemically, and morphologically heterogenous neurodegenerative diseases where the pathology of such diseases can include tauopathy in at least a portion of patients suffering from the disease include, by way of example only, argyrophilic grain disease (AGD), chronic traumatic encephalopathy (CTE), corticobasal degeneration (CBD), frontotemporal dementia and parkinsonism including that subset linked to chromosome 17 (FTDP-17), ganglioglioma, gangliocytoma, lipofuscinosis, lytico-bodigdisease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS) (also referred to as progressive supranuclear palsy—PSP), subacute sclerosing panencephalitis (SSPE), Alzheimer's disease, Lytico-bodig disease, and any other neurodegenerative disease including tauopathy in the either etiology and/or pathology of the disease.

As used herein, the “bis-allylic” position refers to the methylene group of 1,4-diene systems of the polyunsaturated lipid described herein (e.g., the Y substitution positions of the polyunsaturated lipid of Formula (I)). As used herein, the “mono-allylic” position refers to the methylene group adjacent to only one double bond but is not the bis-allylic position (e.g., the X substitution positions of the polyunsaturated lipid of Formula (I)). It is further exemplified in the following structure:

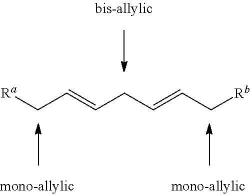

The term “polyunsaturated lipid,” as used herein, refers to a lipid that contains two or more unsaturated bonds, such as double or triple bonds, in its hydrocarbon chain. The polyunsaturated lipid here can be a polyunsaturated fatty acid, polyunsaturated fatty acid ester, polyunsaturated fatty acid thioester, polyunsaturated fatty acid amide, polyunsaturated fatty acid phosphate, or a phospholipid containing the polyunsaturated fatty acid residue.

In some aspects, isotopically modified PUFA molecules may contain one deuterium atom, such as when one of the two hydrogens in a methylene group is replaced by deuterium, and thus may be referred to as a “D1” PUFA. Similarly, an isotopically modified PUFA molecule may contain two, three, four, five, six, seven, eight, nine, ten, eleven, twelve, thirteen or fourteen deuterium atoms, may be referred to as a “D2”, “D3”, “D4”, “D5”, “D6”, “D7”, “D8”, “D9”, “D10”, “D11”, “D12”, “D13” or “D14” PUFA, respectively.

As used herein, the term “D2-arachidonic acid or ester thereof” means that there is deuteration at one or two of the bis-allylic positions of arachidonic acid or ester. Such D2-arachidonic acid or esters include 7,7-D2-arachidonic acid or ester thereof, 10,10-D2-arachidonic acid or ester thereof”, or 13,13-D2-arachidonic acid or ester thereof as well as 7,10-D2-arachidonic acids and related compounds (e.g., 7,13-D2, 10,13-D2-). Mixtures of D2-arachidonic acid or esters thereof are included. Such D2-arachidonic acids or esters can comprise additional deuteration at sites other than the bis-allylic sites such as the allylic sites including up to 6 total deuterium atoms provided that there is a deuterium atom at a bis-allylic site.

As used herein, the term “D4-arachidonic acid or ester thereof” means that there is deuteration at two or three of the bis-allylic positions of arachidonic acid or ester. Such D2-arachidonic acid or esters include 7,7,10,10-D4-arachidonic acid or esters thereof, 10,10,13,13-D4-arachidonic acid or esters thereof”, or 7,7,13,13-D4-arachidonic acid or ester thereof as well as 7,7,10,13-D4-arachidonic acid or esters thereof, and related compounds (7,10,13,13-D4- or 7,10,10,13-D4). Mixtures of D2-arachidonic acid or esters thereof are included. Such D4-arachidonic acids or esters can comprise additional deuteration at sites other than the bis-allylic sites such as the allylic sites including up to 8 total deuterium atoms provided that there are 2 deuterium atoms at two of the bis-allylic sites.

As used herein, the term “D6-arachidonic acid or ester thereof” means that there is di-deuteration at each of the bis-allylic positions of arachidonic acid or ester. Such D6-arachidonic acid or esters are 7,7,10,10,13,13-D6-arachidonic acid or esters thereof. Such D6-arachidonic acid or esters thereof can comprise additional deuteration at sites other than the bis-allylic sites such as the allylic sites including up to 10 total deuterium atoms.

As used herein, “Cₐ to C_{b}” in which “a” and “b” are integers refer to the number of carbon atoms in an alkyl, alkenyl or alkynyl group, or the number of carbon atoms in the ring of a cycloalkyl, aryl, heteroaryl or heterocyclic group. That is, the alkyl, alkenyl, alkynyl, ring of the cycloalkyl, ring of the aryl, ring of the heteroaryl or ring of the heterocyclic can contain from “a” to “b”, inclusive, carbon atoms. Thus, for example, a “C₁ to C₄ alkyl” group or a “C₁-C₄ alkyl” group refers to all alkyl groups having from 1 to 4 carbons, that is, CH₃—, CH₃CH₂—, CH₃CH₂CH₂, (CH₃)₂CH—, CH₃CH₂CH₂CH₂—, CH₃CH₂CH(CH₃)— and (CH₃)₃C—. Likewise, for example, cycloalkyl group may contain from “a” to “b”, inclusive, total atoms, such as a C₃-C₈ cycloalkyl group, 3 to 8 carbon atoms in the ring(s). If no “a” and “b” are designated with regard to an alkyl, cycloalkyl, or cycloalkenyl, the broadest range described in these definitions is to be assumed. Similarly, a “4 to 7 membered heterocycle” group refers to all heterocycle groups with 4 to 7 total ring atoms, for example, azetidine, oxetane, oxazoline, pyrrolidine, piperidine, piperazine, morpholine, and the like.

As used herein, the term “C₁-C₆” includes C₁, C₂, C₃, C₄, C₅ and C₆, and a range defined by any of the two preceding numbers. For example, C₁-C₆ alkyl includes C₁, C₂, C₃, C₄, C₅ and C₆ alkyl, C₂-C₆ alkyl, C₁-C₃ alkyl, etc. Similarly, C₃-C₈ carbocyclyl or cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl. As another example, 3 to 10 membered heterocyclyl groups include 3, 4, 5, 6, 7, 8, 9, or 10 ring atoms, or a range defined by any of the two preceding numbers, such as 4 to 6 membered or 5 to 7 membered heterocyclyl.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyl. The alkyl group may be substituted or unsubstituted.

As used herein, “alkenyl” refers to an alkyl group that contains in the straight or branched hydrocarbon chain one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 4 carbon atoms. The alkenyl group of the compounds may be designated as “C₂₋₄ alkenyl” or similar designations. By way of example only, “C₂₋₄ alkenyl” indicates that there are two to four carbon atoms in the alkenyl chain, i.e., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like. An alkenyl group may be unsubstituted or substituted.

As used herein, “alkynyl” refers to an alkyl group that contains in the straight or branched hydrocarbon chain one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 4 carbon atoms. The alkynyl group of the compounds may be designated as “C₂₋₄ alkynyl” or similar designations. By way of example only, “C₂₋₄ alkynyl” indicates that there are two to four carbon atoms in the alkynyl chain, i.e., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl, and cyclooctyl. A cycloalkyl group may be unsubstituted or substituted.

As used herein, “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene, and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 5 to 10 atoms in the ring(s), 6 to 10 atoms in the ring(s) or 5 to 6 atoms in the ring(s), such as nine carbon atoms and one heteroatom; eight carbon atoms and two heteroatoms; seven carbon atoms and three heteroatoms; eight carbon atoms and one heteroatom; seven carbon atoms and two heteroatoms; six carbon atoms and three heteroatoms; five carbon atoms and four heteroatoms; five carbon atoms and one heteroatom; four carbon atoms and two heteroatoms; three carbon atoms and three heteroatoms; four carbon atoms and one heteroatom; three carbon atoms and two heteroatoms; or two carbon atoms and three heteroatoms. Furthermore, the term “heteroaryl” includes fused ring systems where two rings, such as at least one aryl ring and at least one heteroaryl ring or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazineA heteroaryl group may be substituted or unsubstituted.

As used herein, “heterocyclyl” refers to three-, four-, five-, six-, seven-, eight-, nine and ten-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings (i.e., heterocyclyl groups are not aromatic). The heteroatom(s) is an element other than carbon including, but not limited to, oxygen, sulfur and nitrogen. A heterocycle may further contain one or more carbonyl functionalities, so as to make the definition include oxo-systems such as lactams, lactones, and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” refers to compounds wherein the heterocyclyl contains a linkage of one or more atoms connecting non-adjacent atoms. As used herein, the term “spiro” refers to two rings which have one atom in common and the two rings are not linked by a bridge. Heterocyclyl groups can contain 3 to 10 atoms in the ring(s), 3 to 8 atoms in the ring(s), 3 to 6 atoms in the ring(s), or 5 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heterocyclyl group may be quaternized. Heterocyclyl groups can be linked to the rest of the molecule via a carbon atom in the heterocyclyl group (C-linked) or by a heteroatom in the heterocyclyl group, such as a nitrogen atom (N-linked). Heterocyclyl groups may be unsubstituted or substituted. Examples of such “heterocyclyl” groups include but are not limited to, aziridine, oxirane, thiirane, azetidine, oxetane, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “substituted” that group can be substituted with the above substituents. In some embodiments, substituted group(s) is (are) substituted with one or more substituent(s) individually and independently selected from C₁-C₄ alkyl, C₁-C₄ alkoxy, C₁-C₄haloalkyl, C₁-C₄ halalkoxy, amino, hydroxy, and halogen.

As used herein, the term “thioester” refers to a structure in which a carboxylic acid and a thiol group are linked by an ester linkage or where a carbonyl carbon forms a covalent bond with a sulfur atom —C(═O)SR_{A}, wherein R_{A} may include hydrogen, optionally substituted C₁₋₃₀ alkyl, (branched or straight), optionally substituted C₂₋₃₀ alkenyl (branched or straight), optionally substituted C₂₋₃₀ alknynyl (branched or straight), or optionally substituted ring structure such as C₆₋₁₀ aryl, heteroaryl, carbocyclyl, cycloalkyl or heterocyclyl. “Polyunsaturated fatty acid thioester” refers to a structure P—C(═O)SR_{A}, wherein P is a polyunsaturated fatty acid described herein.

As used herein, the term “amide” refers to compounds or moieties of the structure —C(O)NR_{A}R_{B} and R_{A} and R_{B} can independently be hydrogen, optionally substituted C₁₋₃₀ alkyl (branched or straight), optionally substituted C₂₋₃₀ alkenyl (branched or straight), optionally substituted C₂₋₃₀ alknynyl (branched or straight), or optionally substituted ring structure such as C₆₋₁₀ aryl, heteroaryl, carbocyclyl, cycloalkyl or heterocyclyl. “Polyunsaturated fatty acid amide” refers to a structure a structure P—C(═O)NR_{A}R_{B}, wherein P is a polyunsaturated fatty acid described herein.

As used herein, “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

As used herein, the term “pharmaceutically acceptable salt” as used herein is a broad term, and is to be given its ordinary and customary meaning to a person of ordinary skill in the art (and is not to be limited to a special or customized meaning), and refers without limitation to a salt of a compound that does not cause significant irritation to an organism to which it is administered and does not abrogate the biological activity and properties of the compound.

As used herein, the term “oral dosage form” has its ordinary meaning as understood by those skilled in the art and thus includes, by way of non-limiting example, a formulation of a drug or drugs in a form administrable to a human, including pills, tablets, cores, capsules, caplets, loose powder, solutions, and suspensions.

As used herein, the terms “patient” or “subject” refers to a human patient.

As used herein, the act of “providing” includes supplying, acquiring, or administering (including self-administering) a composition described herein.

As used herein, the term “administering” a drug includes an individual obtaining and taking a drug on their own. For example, in some embodiments, an individual obtains a drug from a pharmacy and self-administers the drug in accordance with the methods provided herein.

The term “therapeutically effective amount” as used herein, refers to an amount of one or more isotopically modified polyunsaturated lipids described herein sufficient to treat, ameliorate a neurodegenerative disease or condition related to tauopathy, or to exhibit a detectable therapeutic effect, for example, to prevent, inhibit, or slow down the progression of a neurodegenerative disease or condition related to tauopathy. The effect may be detected by any means known in the art. In some embodiments, the precise effective amount for a subject can depend upon the subject's body weight, size, and health; the nature and extent of the condition; and the therapeutic or combination of therapeutics selected for administration. Therapeutically effective amounts for a given situation may be determined by routine experimentation that is within the skill and judgment of the clinician.

As used herein, the term “with food” is defined to mean, in general, the condition of having consumed food during the period between from about 1 hour prior to the administration of the isotopically modified compound described herein to about 2 hours after the administration of such compound. In some embodiments, the food is a solid food with sufficient bulk and fat content that it is not rapidly dissolved and absorbed in the stomach. Preferably, the food is a meal, such as breakfast, lunch, or dinner. In some embodiments, the food contains fats, undeuterated PUFAs or esters thereof.

As used herein, the term “reduced rate of disease progression” means that the rate of disease progression is attenuated after initiation of treatment as compared to the patient's natural history. In one embodiment, the reduced rate of disease progression is measured by using the Progressive Supranuclear Palsy Rating Scale or the Unified Parkinson's Disease Rating Scale to determine the rate of disease progression during the natural history and, again, measuring the either score during the interval starting with therapy and again after a set period of time thereafter (e.g., every 3 months). Both rates are then annualized and a reduced rate of disease progression results in a percentage change of at least 30% in either score before and after.

A “therapeutic concentration” means a concentration of a deuterated arachidonic acid that reduces the rate of disease progression by at least 30%. Since obtaining the concentration of a deuterated arachidonic acid in the neurons or in the spinal fluid of a patient is either not feasible or optimal, the therapeutic concentration is based on the concentration of either deuterated linoleic acid or deuterated arachidonic acid found in red blood cells as provided in the Examples below. Accordingly, any reference made herein to a therapeutic concentration of deuterated arachidonic acid is made by evaluating its concentration in red blood cells. In one embodiment, a therapeutic concentration of deuterated arachidonic acid is provided in Table 1 below.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant contains a designed pause in dosing. For example, a dosing schedule that provides dosing 6 days a week is one form of periodic dosing. Another example is allowing the patient to pause administration for from about 3 or 7 or days due to personal reasons provided that the patient is otherwise at least 75 percent compliant.

The term “undeuterated” means that the compounds comprise only the naturally occurring amount of deuterium. The term “deuterated” means that the compounds have been chemically modified to contain more than the naturally occurring amount of deuterium.

In any of the embodiments described herein, methods of treatment can alternatively entail use claims, such as Swiss-type use claims. For example, a method of treating a subject having FRDA can alternatively entail the use of a compound in the manufacture of a medicament for the treatment of FRDA, or a compound for use in the treatment of FRDA.

### Isotopically Modified Polyunsaturated Lipids

In some embodiments, the isotopically modified polyunsaturated lipid comprises a fatty acid, a fatty acid ester, a fatty acid thioester, a fatty acid amide, a fatty acid phosphate, or a phospholipid derivative of the fatty acid, or combinations thereof. In some further embodiments, the phospholipid containing the deuterated polyunsaturated fatty acid residue after an esterification or amidation reaction between the carboxyl group of the fatty acid and the hydroxyl or amino group of the phospholipid.

In some embodiments, the isotopically modified polyunsaturated lipid is a deuterated polyunsaturated fatty acids or derivatives thereof (including but not limited to ester, thioester, amide, phosphate, or phospholipid). In some such embodiments, the polyunsaturated lipid is deuterated at one or more bis-allylic positions. In some such embodiments, the polyunsaturated lipid is deuterated at all bis-allylic positions. In some further embodiments, the polyunsaturated lipids further deuterated at one or more mono-allylic positions. In some embodiments, the deuterated polyunsaturated lipid is deuterated linoleic acid, deuterated linolenic acid, deuterated arachidonic acid, deuterated eicosapentaenoic acid, deuterated docosahexaenoic acid, or a salt or an ester thereof. In some further embodiments, the ester is an alkyl ester, a triglyceride, a diglyceride, or a monoglyceride. In further embodiments, the ester is an ethyl ester.

In some embodiments, the polyunsaturated lipid has a structure of Formula (I):

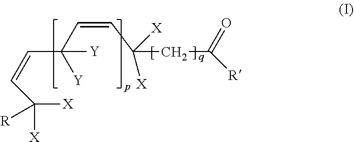

wherein:
R is optionally substituted C₁-C₁₀ alkyl;
R′ is —OR¹, —SR¹, —O(CH₂)CH(OR³)CH₂(OR⁴), —NR⁵R⁶ or

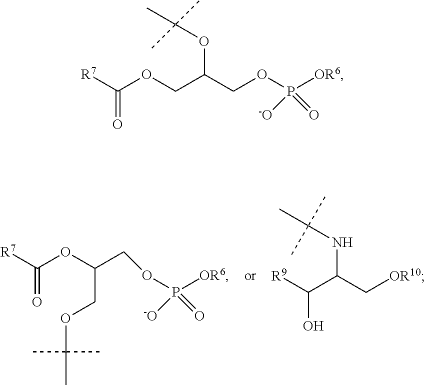

each R¹ and R² is independently H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl; each of R₃ and R₄ is independently H, optionally substituted —C(═O)C₁-C₂₁ alkyl, optionally substituted —C(═O)C₂-C₂₁ alkenyl, or optionally substituted —C(═O)C₂-C₂₁ alkynyl;

each of R⁵ and R⁶ is independently H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl; or R⁵ and R⁶ together with the nitrogen atom to which they are attached form an optionally substituted 3 to 10 membered heterocyclyl;
each R⁷ is independently optionally substituted C₁-C₂₁ alkyl, optionally substituted C₂-C₂₁ alkenyl, optionally substituted C₂-C₂₁ alkynyl;
each R⁸ is independently H, —CH₂CH₂N⁺(CH₃)₃, —CH₂CH₂NH₂, —CH₂CH₂NH₃⁺, —CH₂CH(NH₂)C(═O)O⁻, —CH₂CH(OH)CH₂OH, a mono-saccharide, a di-saccharide, or an oligosaccharide;
R⁹ is optionally substituted C₈-C₂₁ alkyl, optionally substituted C₈-C₂₁ alkenyl, or optionally substituted C₈-C₂₁ alkynyl;
R¹⁰ is H,

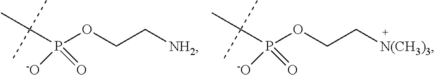

a monosaccharide, a di-saccharide, or an oligosaccharide;
each X and Y is independently H or D, provided that at least one of X and Y is D; and each of p and q is independently an integer of 1, 2, 3, 4, 5, 6, 7, 8, 9 or 10.

In some embodiments, the X substitution position is also referred to as a monoallylic position and the Y substitution position is also referred to as a bis-allylic position. In some such embodiment, at least one Y is D.

In some embodiments of the polyunsaturated lipid of Formula (I), at least one of Y is D (meaning that the polyunsaturated lipid is deuterated at one or more bis-allylic positions). In some further embodiments, each of Y is D (meaning that the polyunsaturated lipid is deuterated at all bis-allylic positions). In some such embodiments, each of X is H. In some other embodiments, at least one X is D (meaning that the polyunsaturated lipid is also deuterated at one or more mono-allylic positions. In some embodiments, R is methyl, C₄ alkyl, or C₇ alkyl, each optionally substituted with one or more D. In other embodiments, R is unsubstituted.

In some embodiments of the polyunsaturated lipid of Formula (I), the polyunsaturated lipid is deuterated linoleic acid or a derivative thereof of Formula (Ia) (where R is n-butyl, p=1, and q=6):

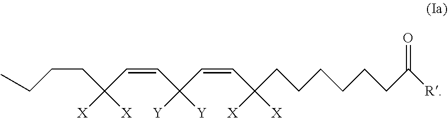

In some such embodiments, one or both Y is D. In some further embodiments, each X is H. In other embodiments, at least one of X is D. In some such embodiments, R′ is —OR¹, wherein R¹ is H or optionally substituted C₁-C₂₁ alkyl. In one embodiment, R¹ is ethyl. In one such embodiment, the deuterated polyunsaturated lipid is 11,11-D2-linoleic acid (D2-Lin), a pharmaceutically acceptable salt thereof, or ethyl ester thereof.

In some embodiments of the polyunsaturated lipid of Formula (I), the polyunsaturated lipid is deuterated linolenic acid or a derivative thereof of Formula (Ib) (where R is methyl, p=2, and q=6):

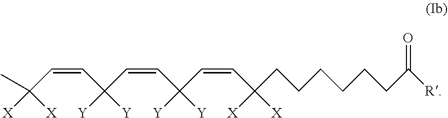

In some such embodiments, at least one Y is D. In some further embodiments, each Y is D. In some further embodiments, each X is H. In other embodiments, at least one of X is D. In some such embodiments, R′ is —OR¹, wherein R¹ is H or optionally substituted C₁-C₂₁ alkyl. In one embodiment, R¹ is ethyl. In one such embodiment, the deuterated polyunsaturated lipid is 11,11, 14, 14-D4-linolenic acid, a pharmaceutically acceptable salt thereof, or ethyl ester thereof.

In some embodiments of the polyunsaturated lipid of Formula (I), the polyunsaturated lipid is deuterated arachidonic acid or a derivative thereof of Formula (Ic) (where R is n-butyl, p=3, and q=2):

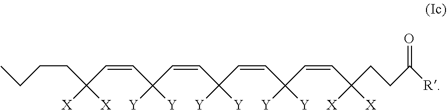

In some such embodiments, at least one Y is D. In some further embodiments, each Y is D. In some further embodiments, each X is H. In other embodiments, at least one of X is D. In some such embodiments, R′ is —OR¹, wherein R¹ is H or optionally substituted C₁-C₂₁ alkyl. In one embodiment, R₁ is ethyl. In one such embodiment, the deuterated polyunsaturated lipid is 7,7,10,10,13,13-D6-arachadonic acid, a pharmaceutically acceptable salt thereof, or ethyl ester thereof.

In some embodiments of the polyunsaturated lipid of Formula (I), the polyunsaturated lipid is deuterated eicosapentaenoic acid or a derivative thereof of Formula (Id)
(where R is methyl, p=4, and q=2):

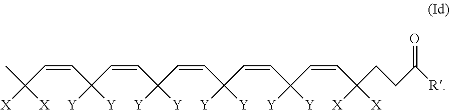

In some such embodiments, at least one Y is D. In some further embodiments, each Y is D. In some further embodiments, each X is H. In other embodiments, at least one of X is D. In some such embodiments, R′ is —OR¹, wherein R¹ is H or optionally substituted C₁-C₂₁ alkyl. In one embodiment, R¹ is ethyl. In one such embodiment, the deuterated polyunsaturated lipid is 7,7,10,10,13,13,16,16-D8-eicosapentaenoic acid, a pharmaceutically acceptable salt thereof, or ethyl ester thereof.

In some embodiments of the polyunsaturated lipid of Formula (I), the polyunsaturated lipid is deuterated docosahexaenoic acid or a derivative thereof of Formula (Ie) (where R is methyl, p=5, and q=1):

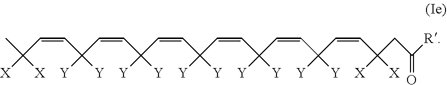

In some such embodiments, at least one Y is D. In some further embodiments, each Y is D. In some further embodiments, each X is H. In other embodiments, at least one of X is D. In some such embodiments, R′ is —OR¹, wherein R¹ is H or optionally substituted C₁-C₂₁ alkyl. In one embodiment, R¹ is ethyl. In one such embodiment, the deuterated polyunsaturated lipid is 6,6,9,9,12,12,15,15,18,18-D10-docosahexaenoic acid, a pharmaceutically acceptable salt thereof, or ethyl ester thereof.

In one embodiment of the compounds of Formula (I) (including (Ia)-(Ie)), the poly-unsaturated lipid is in the form of a glyceride ester, wherein R′=—O(CH₂)CH(OR₃)CH₂(OR₄).
When each of R³ and R⁴ is H, such ester is a mono-glyceride, when only one of R³ and R⁴ is H, such ester is a di-glyceride. When neither R³ or R⁴ is H, such ester is a tri-glyceride.

### Mixture of Deuterated Polyunsaturated Lipids

In some embodiments, the method comprises administering a mixture of polyunsaturated lipids described herein. In some such embodiments, at least one polyunsaturated lipid in the mixture is deuterated at all bis-allylic positions. In some further embodiments, the one or more polyunsaturated lipids in the mixture is further deuterated at one or more mono-allylic positions. In some such embodiments, the mixture of the polyunsaturated lipid comprises two or more species of the same fatty acid described herein or a derivative thereof, where the only difference between the various species is the number of deuterium at the bis-allylic and/or mono-allylic positions. For example, when the mixture comprises deuterated linolenic acid, it may comprises the following species:

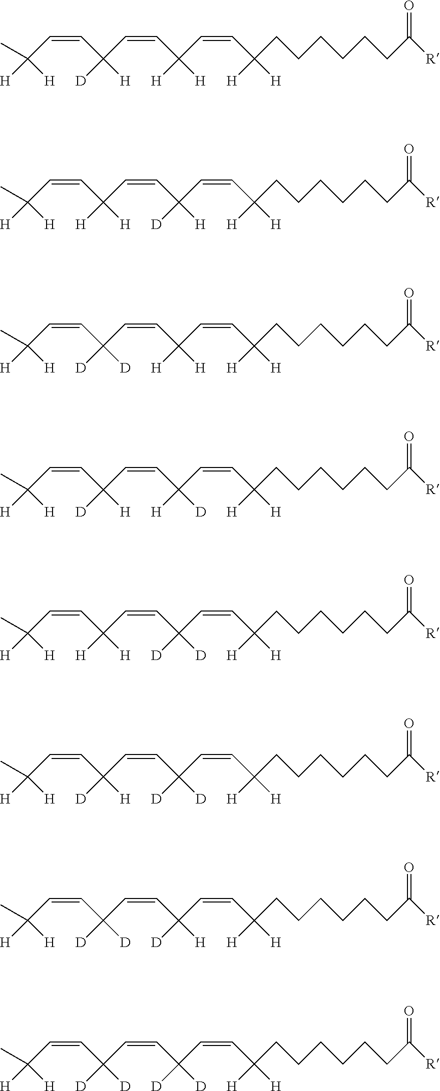

Similarly, when the mixtures comprise species of deuterated linolenic acid or derivative thereof, the mixtures may comprise combinations of various species of linolenic acid, containing any one between one to eight deuterium atoms at various bis-allylic and mono-allylic positions. When the mixture comprises species of deuterated arachidonic acid or derivative thereof, the mixtures may comprise combinations of various species of arachidonic acid, containing any one between one to ten deuterium atoms at various bis-allylic and mono-allylic positions. When the mixture comprises species of deuterated eicosapentaenoic acid or derivative thereof, the mixtures may comprise combinations of various species of eicosapentaenoic acid, containing any one between one to twelve deuterium atoms at various bis-allylic and mono-allylic positions. When the mixture comprises species of deuterated docosahexaenoic acid or derivative thereof, the mixtures may comprise combinations of various species of docosahexaenoic acid, containing any one between one to fourteen deuterium atoms at various bis-allylic and mono-allylic positions.

In some embodiments of the mixture of polyunsaturated lipids described herein, the mixture has a degree of deuteration of at least 50%, for example, at least 50%, 55%, 60%, 65%, 70%, 75%, 80%, 85%, 90%, or 95%. In some further embodiments, the degree of deuteration is at least 70%. The term “deuteration degree”, “degree of deuteration” of “level of deuteration” as used herein, refer to the percentage of deuterium atoms at the bis-allylic positions of a compound as compared to the same compound without deuteration. It may be calculated as following:

degree of deuteration (%)=the number of deuterium atoms at the bis-allylic positions of a compound/the total number of hydrogen and deuterium atoms at the bis-allylic positions of the compound.

For a mixture containing deuterated compounds with various degree of deuteration (e.g., a mixture containing equal amount of Compounds A and B, having degree of deuteration of 33.3% and 66.7% respectively), the total or combined degree of deuteration of the mixture may be calculated as the following:

molar percentage of Compound A*degree of deuteration of Compound A+molar percentage of Compound B*degree of deuteration of Compound B For example, if the mixture contains the following three compounds in equal molar amount:

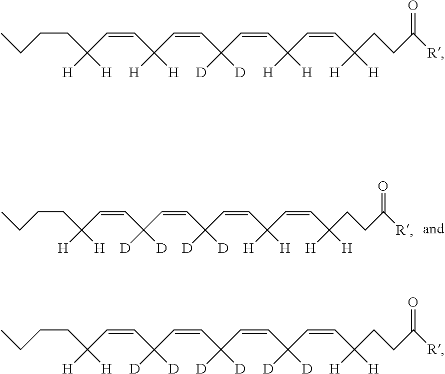

then the degree of deuteration is 66.7%. A more practical way to determine the total percentage of deuteration is to rely on proton-carbon ¹³NMR bis-allylic peak integration measurements and mass-spectrometric methods.

In some embodiments of the method described herein, the first amount of one or more deuterated polyunsaturated lipids is at least about 5% of the total amount of the deuterated polyunsaturated lipids and any undeuterated fats, fatty acids or fatty acid esters administered to, or ingested by the subject. In some other embodiments, the first amount of one or more deuterated polyunsaturated lipids is less than, or about 1% of the total amount of the deuterated polyunsaturated lipids and any undeuterated fats, fatty acids or fatty acid esters administered to, or ingested by the subject.

In some embodiments of the method described herein, the one or more deuterated polyunsaturated lipids are co-administered with at least one antioxidant. In some such embodiments, the antioxidant comprises Coenzyme Q, idebenone, mitoquinone, mitoquinol, vitamin C, or vitamin E, or combinations thereof.

In some embodiments of the method described herein, the deuterated polyunsaturated lipid is incorporated into the subject's tissue (e.g., brain tissue) following administration.

### Methods

The methods of this invention entail the administration of one or more deuterated fatty acids or esters thereof to a patient suffering from a disease modality mediated by tauopathy. Specifically, this invention provides for administration of a sufficient amount of said fatty acid(s) or esters thereof so as to stabilize the neuronal membrane against degradation mediated by abnormal tau.

In one embodiment, administration of a deuterated polyunsaturated fatty acid or ester thereof is maintained so as to ensure that the neurons retain a sufficient amount of these deuterated fatty acids particularly in their membrane notwithstanding the natural turnover of lipids/phospholipids in the membrane. When administered in the manner described herein, patients exhibit a significant reduction in disease progression as shown in the examples.

Such reduction evidence that that the abnormal tau is either cleared or is rendered significantly less toxic in the patients. Regardless, the outcome of such administration is a substantial improvement in protecting the neurons from further damage.

As per Example 8 below, the patients were treated with 11,11-D2-linoleic acid ethyl ester which in vivo is hepatically converted to 13,13-D2-arachidonic acid. Arachidonic acid is highly enriched in the CNS and, in particular, neurons including their cellular membranes. In order to assess a therapeutic concentration of 13, 13-D2-arachidonic acid in these neurons, the clinician can use its concentration in red blood cells as a proxy. In such a situation, a concentration of at least about 3.0% of 13,13-D2-arachidonic relative to the total amount of undeuterated arachidonic acid and deuterated arachidonic acid in the red blood cells evidences a therapeutic concentration of 13,13-D2-arachidonic acid in the neurons. As the amount of deuteration at the bis-allylic sites of arachidonic acid increase, the amount of these more highly deuterated arachidonic acid molecules necessary to achieve a therapeutic result decreases as these will impart greater stability against lipid peroxidation. In general, such therapeutic concentrations are provided below:

TABLE 1

| Deuterated | | | |
|---|---|---|---|
| arachidonic acid | Therapeutic Ratio % | Preferred Ratio % | More Preferably % |
| species | DxAA:(AA + DxAA) | DxAA:(AA + DxAA) | DxAA:(AA + DxAA) |
| D2 arachidonic acid | 3.0% | 5% | 6% or 8% |
| D4 arachidonic acid | 1.0% or 1.5% | 2% or 2.5% | 3% |
| D6 arachidonic acid | 0.5% or 0.75% | 1% or 1.5% | 3% |

The dosing schedules below are designed to achieve a therapeutic concentration of deuterated arachidonic acid as measured by proxy in red blood cells.

### Doses and Dosing Schedule

The following provides a dosing schedule for treating patients suffering from tauopathy.

In some embodiments of the methods described herein, the dosing regimen is provided in a primer period and a maintenance period. The primer period (first dosing period) is designed to minimize the amount of time required to provide a sufficient concentration of deuterated polyunsaturated fatty acids (PUFAs) in the neurons to stabilize them from oxidative damage arising from abnormal tau. The maintenance dose is designed to maintain a therapeutic concentration of the deuterated PUFAs in the neurons.

### Primer Dosing

The primer dosing constitutes a loading amount of a deuterated PUFAs in the neurons so as to minimize the amount of time between start of treatment and when neuronal stability is reached. In some embodiments, the daily or periodic amount of one or more deuterated PUFAs administered to the patent is about 0.1 g, 0.2 g, 0.5 g, 1.0 g, 1.5 g, 2.0 g, 2.5 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g 18 g, 18.5 g, 19 g, 19.5 g, or 20 g, or a range defined by any of the two preceding values. In some embodiments, the first amount of one or more deuterated polyunsaturated lipids administered to the subject is from about 0.1 g to about 20 g, from about 1 g to about 10 g, from 2 g to about 5 g. In one embodiment, the amount is about 3 g or 2.88 g. In some embodiments, the amount administered in the primer dose is about 6 g or 5.78 g.

In some embodiments, the one or more deuterated polyunsaturated lipids administered are in a unit dosage form. The unit dose may constitute one pill, tablet, or otherwise ingestible form of a pharmaceutical dose. In some other embodiments, the deuterated polyunsaturated lipid(s) is provided in 1 gram pills or capsules. For example, where a dose is about 3 g, it may be provided in the form of 1 to 6 tablets or capsules, each containing about 0.5 g or about 1 g of the deuterated polyunsaturated lipid(s). In one embodiment, the first amount is about 3 g administered in 3 capsules, with each capsule contains about 1 g of the deuterated fatty acid or fatty acid ester. In another embodiment, the first amount is about 2.88 g administered in 3 capsules, with each capsule contains about 0.96 g of the lipid(s).

In some embodiments of the method described herein, the deuterated polyunsaturated lipid(s) may be administered daily or periodically. In some embodiments, the daily or periodic dose may be administered, one or two or more times per day, for example, twice a day, or three times a day. In some such embodiments, the amount of the lipid(s) administered per day is from about 3 g to about 20 g, from about 4 g to about 15 g, or from about 5 g to about 10 g. In one embodiment, the amount of the lipid(s) administered per day is from about 9 g or 8.64 g, administered as three capsules three times a day (with each capsule contains 3 g or 2.88 g of the lipid(s)).

In a preferred embodiment, the primer dose constitutes a daily or periodic dose of about 3 to 9 grams of the deuterated PUFA. More preferably, the daily or periodic primer dose is about 5 g to about 7 g.

In some embodiments of the method described herein, the primer dose is continued for at about 1 week, 2 weeks, 3 weeks, 4 weeks, 5 weeks, 6 weeks, 7 weeks, 8 weeks, or 12 weeks (3 months). In one embodiment, the first period of time is about three months.

By completion of the first dosing period, the concentration of all deuterated fatty acids in the liposomal lipid bilayers of at least 20% (e.g., deuterated linoleic acid and deuterated arachidonic acid) provides for a therapeutic concentration. In a preferred embodiment, the concentration of deuterated fatty acids in the liposomal lipid bilayers is from about 20% to about 60% and, more preferably, from about 20% to about 40%.

### Second Dosing Period

In some embodiments of the method described herein, the dosing method further comprises administering a second or maintenance dose of one or more deuterated polyunsaturated lipids. In some such embodiments, the second dosing period of time starts immediately after the first period of time ends. In some such embodiments, the second period of time is longer than the first period of time. In some such embodiments, the second amount administered per day is about 30 to 70% less than the first amount administered per day. The purpose of the maintenance dose is to provide sufficient deuterated PUFA to the patient to maintain the therapeutic concentration of deuterated fatty acids in the liposomal lipid bilayers is from about 20% to about 60% and, more preferably, from about 20% to about 40%.

In some embodiments, a maintenance effective amount of the one or more deuterated polyunsaturated lipids administered to the subject is about 30 to 70% of the primer dose and more preferably about 35 to about 65% of the primer dose. Examples of suitable maintenance doses are 0.1 g, 0.2 g, 0.5 g, 1.0 g, 1.5 g, 2.0 g, 2.5 g, 3.0 g, 3.5 g, 4.0 g and every half gram increment up to 14 g. Preferred maintenance doses range from about 0.1 g to about 10 g or from 2 g to about 5 g. In one embodiment, the second amount is about 3 g or 2.88 g.

In some embodiments, the maintenance dose of deuterated polyunsaturated lipids is administered are in a single unit dosage form. In some other embodiments, the deuterated polyunsaturated lipid(s) is in two or more unit dosage forms (i.e., a divided dose). In some embodiments, the unit dosage form is a tablet, a capsule, a pill, or pellets. In some further embodiment, the unit dosage form for oral administration, i.e., oral dosage form. For example, where the maintenance dose is about 3 g, it may be provided in the form of 1 to 6 tablets or capsules, each containing about 0.5 g to 3 g of the deuterated polyunsaturated lipid(s). In one embodiment, the second amount is about 3 g administered in 3 capsules, with each capsule contains about 1 g of the lipid(s). In another embodiment, the second amount is about 2.88 g administered in 3 capsules, with each capsule contains about 0.96 g of the lipid(s).

In some embodiments of the method described herein, the maintenance dose of one or more deuterated polyunsaturated lipids may be administered for a second period of at least 1 month, 2 months, 3 months, 4 months, 5 months, 6 months, 7 months, 8 months, 9 months, 10 months, 11 months, 1 year or for the remaining lifespan of the patient.

In some embodiments of the method described herein, the first amount and/or the second amount of the one or more polyunsaturated lipids described herein may be administered with food (e.g., breakfast, lunch or dinner) or immediately after a meal. In some such embodiments, the subject may also ingest one or more non-isotopically modified polyunsaturated lipids, either concurrently, prior to, or subsequent to the administration of the deuterated polyunsaturated lipid(s) described herein. In some embodiments, the first amount of one or more deuterated polyunsaturated lipids is at least about 5%, 10%, 15%, 20%, 25%, 30%, 35%, 40%, 45%, 50% or greater of the total amount of the deuterated polyunsaturated lipid(s) and any undeuterated fats, fatty acids or fatty acid esters administered to, or ingested by the subject delivered the subject. In some other embodiments, the amount of 11,11-D2-linoleic acid or the ester thereof is equal to less than about 5%, 4%, 3%, 2%, 1%, or 0.5% of the total amount of the polyunsaturated fatty acids and polyunsaturated fatty acid esters delivered to the subject. In some such embodiments, the non-isotopically modified PUFA or derivatives thereof may be taken concurrently, prior to, or subsequent to the administration of isotopically modified PUFA.

In some embodiments of the method described herein, the one or more isotopically modified polyunsaturated lipids described herein may be administered with at least one antioxidant. In some such embodiments, the antioxidant is selected from the group consisting of Coenzyme Q, idebenone, mitoquinone, mitoquinol, vitamin E, and vitamin C, and combinations thereof. In some such embodiments, the at least one antioxidant may be taken concurrently, prior to, or subsequent to the administration of the polyunsaturated lipid(s) thereof. In some embodiments, the antioxidant and the polyunsaturated lipid(s) may be in a single dosage form. In some embodiments, the single dosage form is selected from the group consisting of a pill, a tablet, and a capsule.

In one preferred first and second dosing regimens of 11,11-D2-linoleic acid ethyl ester include the following:

| First (Primer) dose | Second (Maintenance) dose |
|---|---|
| about 9 grams (e.g., 8.64 g) | about 6 grams |
| about 9 grams (e.g., 8.64 g) | about 5 grams |
| about 6 grams (e.g., 8.64 g) | about 4 grams |
| about 6 grams (e.g., 5.88 g) | about 3 grams |
| about 4 grams (e.g., 3.92 g) | about 3 grams |
| about 4 grams (e.g., 3.92 g) | about 2 grams |

In one preferred first and second dosing regimens of 13,13-D2-arachidonic acid ethyl ester include the following:

| First (Primer) dose | Second (Maintenance) dose |
|---|---|
| about 6 grams | about 4 grams |
| about 6 grams | about 3.5 grams |
| about 4.5 grams | about 3 grams |
| about 4.5 grams | about 2.5 grams |
| about 3 grams | about 2 grams |
| about 3 grams | about 1.5 grams |

In one preferred first and second dosing regimens of 7,7-10,10-D4-arachidonic acid ethyl ester or 7,7,13,13-D4-arachidonic acid ethyl ester or 10,10,13,13-D4-arachidonic acid ethyl ester include the following:

| First (Primer) dose | Second (Maintenance) dose |
|---|---|
| about 3 grams | about 2 grams |
| about 3 grams | about 1.5 grams |
| about 2 grams | about 1 gram |
| about 2 grams | about 0.75 grams |
| about 1 gram | about 0.66 grams |
| about 1 gram | about 0.5 grams |

In one preferred first and second dosing regimens of 7,7,10,1013,13-D6-arachidonic acid ethyl ester include the following:

| First (Primer) dose | Second (Maintenance) dose |
|---|---|
| about 2.5 grams | about 1.5 grams |
| about 2 grams | about 0.5 to about 1.5 grams |
| about 1.5 grams | about 0.5 to about 1 gram |
| about 1.0 gram | about 0.4 to about 0.8 grams |
| about 0.75 grams | about 0.5 grams |
| about 0.5 grams | about 0.25 grams |

In some embodiments, the attending clinician can adjust the primer dose upwards as warranted by the condition of the patient. For example, a patient initially on about 6 grams of the 11,11-D2-linoleic acid ethyl ester can have the primer dose increased to about 9 grams day based on the condition of the patient and the judgement of the attending clinician.

In some embodiments, the primer dose is continued for a period of 25 to 120 days and preferably 30 days (1 month) or 90 days (3 months).

In some embodiments, the maintenance dose is continued for the remaining lifespan of the patient.

### Pharmaceutical Compositions

Some embodiments include pharmaceutical compositions comprising: (a) an effective amount of one or more isotopically modified polyunsaturated lipids described herein, or a pharmaceutically acceptable salt thereof; and (b) a pharmaceutically acceptable carrier, diluent, excipient or combination thereof. In some embodiments, the polyunsaturated lipid is 11,11-D2-linoleic acid or an ester thereof. In one particular embodiment, the polyunsaturated lipid is 11,11-D2-linoleic acid ethyl ester.

It is also contemplated that it may be useful to formulate the polyunsaturated lipid as a salt form. For example, the use of salt formation as a means of tailoring the properties of pharmaceutical compounds is well known. See Stahl et al., Handbook of pharmaceutical salts: Properties, selection and use (2002) Weinheim/Zurich: Wiley-VCH/VHCA; Gould, Salt selection for basic drugs, Int. J. Pharm. (1986), 33:201-217. Salt formation can be used to increase or decrease solubility, to improve stability or toxicity, and to reduce hygroscopicity of a drug product.

Formulation of polyunsaturated lipid(s) as salt(s) includes, but is not limited to, the use of basic inorganic salt forming agents, basic organic salt forming agents, and salt forming agents containing both acidic and basic functional groups. Various useful inorganic bases for forming salts include, but are not limited to, alkali metal salts such as salts of lithium, sodium, potassium rubidium, cesium, and francium, and alkaline earth metal salts such as berylium, magnesium, calcium, strontium, barium, and radium, and metals such as aluminum. These inorganic bases may further include counterions such as carbonates, hydrogen carbonates, sulfates, hydrogen sulfates, sulfites, hydrogen sulfites, phosphates, hydrogen phosphates, dihydrogen phosphates, phosphites, hydrogen phosphites, hydroxides, oxides, sulfides, alkoxides such as methoxide, ethoxide, and t-butoxide, and the like. Various useful organic bases for forming salts include, but are not limited to, amino acids, basic amino acids such as arginine, lysine, ornithine and the like, ammonia, alkylamines such as methylamine, ethylamine, dimethylamine, diethylamine, trimethylamine, triethylamine and the like, heterocyclic amines such as pyridine, picoline and the like, alkanolamines such as ethanolamine, diethanolamine, triethanolamine and the like, diethylaminoethanol, dimethylaminoethanol, N-methylglucamine, dicyclohexylamine, N,N′-dibenzylethylenediamine, ethylenediamine, piperazine, choline, trolamine, imidazole, diolamine, betaine, tromethamine, meglumine, chloroprocain, procaine, and the like.

Pharmaceutically acceptable salts are well known in the art and include many of the above-recited inorganic and organic bases. Pharmaceutically acceptable salts further include salts and salt-forming agents found in drugs approved by the Food and Drug Administration and foreign regulatory agencies. Pharmaceutically acceptable organic cations for incorporation include, but are not limited to, benzathine, chloroprocaine, choline, diethanolamine, ethylenediamine, meglumine, procaine, benethamine, clemizole, diethylamine, piperazine, and tromethamine. Pharmaceutically acceptable metallic cations for incorporation include, but are not limited to, aluminum, calcium, lithium, magnesium, potassium, sodium, zinc, barium, and bismuth. Additional salt-forming agents include, but are not limited to, arginine, betaine, carnitine, diethylamine, L-glutamine, 2-(4-imidazolyl)ethylamine, isobutanolamine, lysine, N-methylpiperazine, morpholine, and theobromine.

In addition to the selected compound useful as described above, some embodiments include compositions containing a pharmaceutically-acceptable carrier. The term “pharmaceutically acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Pharmaceutically-acceptable carriers include, for example, solid or liquid fillers, diluents, hydrotropies, surface-active agents, and encapsulating substances. Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Per-oral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives or supplements. For example, the pharmaceutical composition is administered concomitantly with one or more antioxidants. In some embodiments, the antioxidant is selected from the group consisting of Coenzyme Q, idebenone, mitoquinone, mitoquinol, vitamin E, and vitamin C, and combinations thereof. In some such embodiments, at least one antioxidant may be taken concurrently, prior to, or subsequent to the administration of 11,11-D2-linoleic acid or the ester thereof. In some embodiments, the antioxidant and 11,11-D2-linoleic acid or the ester thereof may be in a single dosage form. In some embodiments, the single dosage form is selected from the group consisting of a pill, a tablet, and a capsule.

It will be understood by those of skill in the art that numerous and various modifications can be made without departing from the spirit of the present invention. Therefore, it should be clearly understood that the embodiments of the present invention disclosed herein are illustrative only and are not intended to limit the scope of the present invention. Any reference referred to herein is incorporated by reference for the material discussed herein, and in its entirety.

### Co-Administration

In some embodiments, the polyunsaturated lipid(s) disclosed herein are administered in combination with one or more antioxidants.

Although antioxidants cannot cancel the negative effects of PUFA peroxidation due to the stochastic nature of the process and the stability of PUFA peroxidation products (reactive carbonyls) to antioxidant treatment, co-administration of antioxidants with compositions resistant to oxidation, such as those described herein, may prove beneficial for treating oxidative stress-related disorders.

Certain antioxidants contemplated as useful for co-administration include the following: vitamins, such as vitamin C and vitamin E; glutathione, lipoic acid, uric acid, carotenes, lycopene, lutein, anthocyanins, oxalic acid, phytic acid, tannins, coenzyme Q, melatonin, tocopherols, tocotrienols, polyphenols including resveratrol, flavonoids, selenium, eugenol, idebenone, mitoquinone, mitoquinol, ubiquinone, Szeto-Schiller peptides, and mitochondrial-targeted antioxidants. When not explicitly mentioned, quinone derivatives of the aforementioned antioxidants are also contemplated as useful for co-administration.

Some additional embodiments of the present disclosure relate to kits comprising a pharmaceutical composition, prescribing information, and a container, wherein the pharmaceutical composition comprises a therapeutically effective amount of one or more isotopically modified polyunsaturated lipids described herein. In some embodiments, isotopically modified polyunsaturated lipid is a deuterated polyunsaturated acid (PUFA) or an ester, thioester, amide, phosphate, or other prodrug thereof (such as a phospholipid derivative). In some further embodiment, the deuterated PUFA is 11,11-D2-linoleic acid and/or an ester thereof. In one particular embodiment, the isotopically modified PUFA is 11,11-D2-linoleic acid ethyl ester. In some embodiments, the prescribing information advises a subject to take the pharmaceutical composition with food or take the pharmaceutical composition between meals. The kit may include one or more unit dosage forms comprising 11,11-D2-linoleic acid or the ester thereof. The unit dosage forms may be of an oral formulation. For example, the unit dosage forms may comprise pills, tablets, or capsules. The kit may include a plurality of unit dosage forms. In some embodiments, the unit dosage forms are in a container. In some embodiments, the dosage forms are single oral dosage forms comprising 11,11-D2-linoleic acid or the ester thereof, e.g., the ethyl ester.

The methods, compositions and kits disclosed herein may include information. The information may be in a form prescribed by a governmental agency regulating the manufacture, use, or sale of pharmaceuticals, which notice is reflective of approval by the agency of the form of the drug for human or veterinary administration. Such information, for example, may be the labeling approved by the U.S. Food and Drug Administration for prescription drugs, or the approved product insert. The information can include required information regarding dose and dosage forms, administration schedules and routes of administration, adverse events, contraindications, warning and precautions, drug interactions, and use in specific populations (see, e.g., 21 C.F.R. § 201.57 which is incorporated herein by reference in its entirety), and in some embodiments is required to be present on or associated with the drug for sale of the drug. In some embodiments, a kit is for sale of a prescription drug requiring the approval of and subject to the regulations of a governmental agency, such as the Food and Drug Administration of the United States. In some embodiments, the kit comprises the label or product insert required by the agency, such as the FDA, for sale of the kit to consumers, for example, in the U.S. In preferred embodiments, the information instructs an individual to take 11,11-D2-linoleic acid or the ester thereof between meals, or with food, in order to reduce possible adverse event(s), for example gastrointestinal adverse event(s).

Instructions and/or information may be present in a variety of forms, including printed information on a suitable medium or substrate (e.g., a piece or pieces of paper on which the information is printed), computer readable medium (e.g., diskette, CD, etc. on which the information has been recorded), or a website address that may be accessed via the internet. Printed information may, for example, be provided on a label associated with a drug product, on the container for a drug product, packaged with a drug product, or separately given to the patient apart from a drug product, or provided in manner that the patient can independently obtain the information (e.g., a website). Printed information may also be provided to a medical caregiver involved in treatment of the patient. In some embodiments, the information is provided to a person orally.

Some embodiments comprise a therapeutic package suitable for commercial sale. Some embodiments comprise a container. The container can be in any conventional shape or form as known in the art which is made of a pharmaceutically acceptable material, for example a paper or cardboard box, a glass or plastic bottle or jar, a re-sealable bag (e.g., to hold a “refill” of tablets for placement into a different container), or a blister pack with individual dosages for pressing out of the pack according to a therapeutic schedule. The container employed can depend on the exact dosage form involved, e.g., a conventional cardboard box would not generally be used to hold a liquid suspension. It is feasible that more than one container can be used together in a single package to market a single dosage form. For example, tablets may be contained in a bottle which is in turn contained within a box.

The information can be associated with the container, for example, by being: written on a label (e.g., the prescription label or a separate label) adhesively affixed to a bottle containing a dosage form described herein; included inside a container as a written package insert, such as inside a box which contains unit dose packets; applied directly to the container such as being printed on the wall of a box; or attached as by being tied or taped, e.g., as an instructional card affixed to the neck of a bottle via a string, cord or other line, lanyard or tether type device. The information may be printed directly on a unit dose pack or blister pack or blister card.

In one optional exclusionary component, the neurodegenerative disease or condition is not Alzheimer's disease, Parkinson's disease, or frontotemporal dementia.

### EXAMPLES

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

### Abbreviations

The following abbreviations are used in the examples below and throughout the application. These abbreviations have the following meanings. If not defined, the abbreviations have their accepted scientific/medical meaning.
BM=Bone marrow
cm²=Centimeters squared
D2-ADA=13,13-D2-Arachadonic acid
D2-Lin=11,11-D2-Linoleic acid
D2-PUFAs=Polyunsaturated Fatty Acid having 2 deuterium atoms at a bis-allylic methylene
DNA=Deoxynucleic acid
ER=Endoplasmic reticulum
g=grams
GSH=Glutathione
4-HNE=4-hydroxynonenal
Lin=Linoleic acid
H2-LA=Non-deuterated linoleic acid
LPO=Lipid Peroxidation
MAM=Mitochondrial-associated ER membrane
MCB=Monochlorobimane
MMP or ΔΨₘ=Mitochondrial membrane potential
MSC=Mesenchymal Stem Cells
mV=Millivolts
nM=Nanomolar
nm=Nanometers
PSP=Progressive supranuclear Palsy
PSPRS=Progressive Supranuclear Palsy Rating Scale
ROS=Reactive oxygen species
SEM=Standard error of the mean
SOD1=Free radical scavenging enzyme-1
SOD2=Free radical scavenging enzyme-2
SROS=Steele-Richardson-Olszewski Syndrome (progresssive supranuclear palsy—PSP)
TMRM=tetramethylrhodamine methyl ester
μM=Micromolar
μm=microns
UPDRS=Unified Parkinson's Disease Rating Scale
   Reversal of Reactive Oxygen Species Generation and Lipid Peroxidation in Mesenchymal Stem Cells Derived from Patients with SROS

The effect of D2-Lin on mesenchymal stem cells derived from patients with SROS was assessed using non-deuterated Lin as a control. MSC preparations from bone marrow aspirates from control subjects were compared to 3 subjects with SROS, following protocols described in Hill S., et al., Free Radic. Biol. Med. (2012) 53:893-906; and Cotticelli M G., et al., Redox Biol. (2013) 1:398-404.

MSCs derived from patients with SROS exhibit characteristics of reduced mitochondrial health when compared to MSCs from age-matched control subjects. These include changes in mitochondrial morphology, number, and function. Incubation of these SROS MSCs with D2-PUFAs for 72 hours restores these parameters to control MSC levels.

The structural and functional changes in mitochondria derived from SROS subjects were associated with increased rates of LPO, and coincident reductions in glutathione levels. These changes were also reversed with D2-Lin pre-treatment. Glutathione deficiency associated with Parkinson's disease and SROS has been recognized previously. Fitzmaurice, et al., Movement Disorders, (2003) 18:969-976. Furthermore, SROS-related LPO was reported previously. However, D2-Lin experiments reported here provide for the first time an explicit link between GSH depletion as a consequence of the elevated LPO level. Furthermore, the glutathione-LPO axis is of particular relevance to the SROS cell recovery for it overlaps with a novel cell death modality termed ferroptosis, an LPO-driven non-apoptotic cell death mechanism. Sun, et al., Cell Death Disease (2018) 9:753-768.

### Example 1. D2-Lin Effects on Mitochondrial Membrane Potential

MMP is an important indicator of mitochondrial health and is typically kept at low values (100-140 mV). A long-lasting drop or rise of normal MMP levels may induce loss of cell viability and various pathologies. ROS and LPO levels are directly linked to MMP, and increase exponentially at MMP>140 mV. Zorova et al., Anal. Biochem. (2018) 552:50-59. Using a fluorescent indicator of MMP, SROS-MSCs showed significantly increased basal MMP compared to control MSCs. Incubation of the cells with D-PUFAs for 72 hours had no effect on MMP for the controls but it reduced mitochondrial membrane potential in the SROS cells from the SROS patients.

### Example 2. D2-Lin Effect on Mitochondrial Structure

Presynaptic terminals typically contain multiple mitochondria. Misshaped mitochondria can clog the pathways that transport them along neurons and through long axons and elaborate dendritic arbors. Mattson et al., Neuron (2009) 60:748-766. Accordingly, mitochondrial shape is another characteristic of mitochondrial health. Also, the communication pathway between ER and mitochondria through MAM is compromised in misshaped mitochondria, further disturbing protein folding, lipid metabolism, Cₐ²⁺ homeostasis and apoptosis. Lee et al., Mol. Cells (2018) 41:1000-1007. Coincident with the improvements in MMP with D-PUFA incubation, the abnormalities of mitochondrial shape seen in SROS MSCs were improved with D-PUFA pre-treatment, resulting in more uniformly looking mitochondria.

### Example 3. D2-Lin Effect on Mitochondrial Number

The number of mitochondria within the cells is regulated by the balance between mitochondrial biogenesis and mitophagy. Accordingly, for the normal maintenance of the mitochondrial balance, SROS-MSCs should activate mitochondrial biogenesis. However, the level of mitochondrial DNA in the cells, measured by PicoGreen fluorescence in non-nuclear area as an estimation of the number of mitochondria, was significantly lower in SROS-MSC (67.5%), compared to control MSC. Various factors, for example excessive fusion can result in a lower number of mitochondria in the cell, leading to impaired respiration, a lower production of ATP, and increased oxidative damage. Arun et al., Curr. Neuropharmacol. (2016) 14:143-154. However, after 72 hours incubation of the cells with D2-PUFAs mitochondrial DNA increased for both control and SROS MSCs. This was particularly notable in the SROS MSCs, where the level increased even higher than it did in the control (from 67.5% to 105.4 vs. 100% to 109%), strongly suggesting that D2-PUFA completely restored mitochondrial numbers in MSC.

### Example 4. D2-Lin Effect on Mitochondrial Function

While the cause of SROS is unknown many genes encoding proteins important in mitochondrial function have been implicated, including SOD1 and SOD2, which explains mitochondrial dysfunction and excess ROS production as well as elevated LPO. Angelova et al., FEBS Lett. 2018; 592:692-702. The production of mitochondrial reactive oxygen species (ROS) is linked to the rate of respiration, and this parameter can be measured using the mitochondria-specific probe MitoTracker Red CM-H2xROS, which fluoresces upon oxidation. Significant basal differences between the control and SROS MSCs were seen; these differences were reversed when the MSCs were incubated with D2-Lin (Rate of mitotracker ROS fluorescence, %: P1, 169 (control)−106 (D2-Lin); P2, 288 (control)−138 (D2-Lin), P3, 308 (control)−124 (D2-Lin)).

### Example 5. D2-Lin Effect on Lipid Peroxidation

LPO has been implicated as an early and pivotal factor in SROS. Specifically, increased levels of n-6 PUFA oxidation products such as 4-HNE were reported, as a direct consequence of the LPO chain reaction. Accordingly, the difference in basal MMP and ROS generation between control and the 3 cell lines of SROS MSCs was associated with increased rates of LPO, as detected by the LPO-specific probe, BODIPY C11. SROS MSCs had a significantly higher rate of lipid peroxidation (161.8±8.2% of control; N=7; p<0.001). Again, a 72-hour incubation of the cell lines with D2-Lin restored the SROS MSCs to normal levels. The rate of LPO in control MSCs was reduced to 84.3% of the pre-treatment value, and effect was even more pronounced in SROS MSCs (161.8% to 109.8%; N=8).

### Example 6. D2-Lin Effect on Glutathione

Glutathione-related machinery is the major mechanism involved with slowing down the inhibition/propagation the LPO chain as well as mopping up the toxic end products of LPO. Consumption of the endogenous antioxidant glutathione is an indirect measure of oxidative stress. Using monochlorobimane (MCB) to measure the level of GSH, mitochondrial ROS overproduction in SROS-MSCs significantly decreased the level of GSH compared to control MSCs. Incubation with non-antioxidant D2-PUFAs significantly increased level of GSH in SROS MSCs, but not in control lines. This observation suggests that the effect of D-PUFAs on GSH is due to modulatory effect of these lipids on mitochondrial metabolism which reduced mitochondrial ROS overproduction.

The following in vitro and in vivo examples utilize PSP as a representative disease involving tauopathy. Other diseases involving tauopathy can be treated by the methods described therein.

### Example 7. In Vitro Results

MSC preparations from BM were obtained from control subjects and from the PSP subjects following previously described protocols [14-18]. In short, MSCs were isolated from BM aspirates seeding 50,000 mononucleated cells/cm2 in αMEM (Thermo Fisher Scientific, Waltham, MA, USA) supplemented with 10% fetal bovine serum (FBS; Thermo Fisher Scientific), in T75 flasks. The cultures were incubated at 37° C., 20% O₂, 5% CO₂. Medium changes were performed twice a week. Two weeks after initial seeding, primary MSC colonies were detached with a 10-minute incubation at 37° C. with TrypLE Select Enzyme (Thermo Fisher Scientific) and replated at 4000 cells/cm² in the same medium. MSC identity was previously assessed. Subsequent passages were performed following the same steps. Passage 4-6 MSCs were used for all experiments. BM from PSP patients was collected in the context of a clinical protocol authorized by the local Ethics Committee of Fondazione IRCCS Ca′ Granda Ospedale Maggiore Policlinico (Italy), by the national competent authority for phase-I cell therapy at the National Health Institute (Istituto Superiore di Sanith) and approved by the Italian Medicines Agency (Agenzia Italiana del Farmaco, AIFA). The trial is registered at ClinicalTrials.gov (NCT01824121). All BM donors gave their written informed consent.

### Live Cell Imaging

Lipid peroxidation was measured using confocal microscopy (Zeiss 710 LSM with an integrated META detection system). The rate of lipid peroxidation was measured using C11-BODIPY 581/591 (2 M; Molecular probes) which was excited by the 488 and 543 nm laser line and fluorescence measured using a band-pass filter from 505 to 550 nm and 560 nm long-pass filter (40× oil-objective). Illumination intensity was kept to a minimum (0.1-0.2% of laser output) to prevent phototoxicity and the pinhole was set to give an optical slice of ˜2 m. Addition of a bright-field image allowed separation between neurons and glia that are visibly different and are situated on different focal planes.

For assessments of glutathione levels, the PSP-MSCs cultures were incubated with 50 μM monochlorobimane (MCB) (Molecular Probes, Invitrogen) for 40 minutes in HEPES buffered salt solution prior to imaging. Cells were then washed with HEPES buffered salt solution and images of the fluorescence of the MCB-GSH were acquired using a Zeiss 710 CLSM with excitation at 405 nm and emission at 435-485 nm. Mitochondrial ROS generation rate was assessed using MitoTracker® Red CM-H2XRos (Thermo Fisher Scientific) which accumulates into mitochondria upon oxidation. The fluorescence measurement was obtained by excitation with 561 nm laser and emission detected above 580 nm. Mitochondrial membrane potential (ΔΨm) was assessed using 25 nM tetramethylrhodamine methyl ester (TMRM, Thermo Fisher Scientific) at 560 nm excitation and fluorescence was measured above 580 nm. Z-stack images were collected and the fluorescence intensity of TMRM was analyzed using Zen software (Zeiss).

D2-Lin effects on lipid peroxidation, mitochondrial function, glutathione, mitochondrial membrane potential, mitochondrial number, and mitochondrial structure were compared to the effects on MSCs derived from healthy control age-matched subjects. H2-LA and D2-Lin were added to cultures as described previously [17].

Using the LPO-specific probe BODIPY C11, PSP MSCs had a significantly higher rate of LPO compared to controls. After a 72-hour incubation of the cell lines with D2-Lin, PSP MSCs returned to normal levels (treated), while PSP MSCs incubated with non-deuterated linoleic acid (H2-LA) (control) remained elevated at over two times that of the treated levels.

Glutathione levels were measured using MCB. The MCB intensity was reduced in PSP MSCs compared to HC. After incubation with D2-Lin (test), glutathione levels were restored to HC levels, while glutathione levels remained low in PSP MSCs after incubation with H2-LA or about 30% less than those of found in the test.

### Example 8. In Vivo Results

Three patients diagnosed with likely PSP underwent baseline assessment using the 28-item Progressive Supranuclear Palsy Rating Scale (PSPRS) [19] and the Unified Parkinson's Disease Rating Scale (UPDRS) [20,21]. They were then treated with D2-Lin (2.88 g BID; 5.76 g total daily dose) and observed for disease progression. The second male patient had his dose increased (2.88 g TID; 8.64 g total daily dose) after the first year of treatment. During the treatment period, scores in the 2 rating scales were determined every 3 months. Pharmacokinetic (PK) sampling was performed at month 3. These analytes included plasma and RBC membrane levels of (D2-LA) and its centrally active metabolite D2-AA.

The three patients were 2 males (age 66 and 73) and one female (age 74) each of whom had pre-treatment symptom duration of 6 years and 3 years for the two males and 2 years for the female. The baseline PSPRS for the two males was 17 and 12 respectively and 13 for the female. The baseline UPDRS for the two males was 44 and 36 respectively and 21 for the female.

After 3 months of therapy, the slope of the PSPRS changed from the historical decline of 0.91 points/month to a mean of decline of 0.16 points/month (+/−0.23 SEM). The UPDRS slope changed from an expected increase of 0.95 points/month to an average increase in score of 0.28 points/month (+/−0.41 SEM).

At 3 months, the following data was collected:

| Patient | % ratio of 11,11-D2-AA to AA |
|---|---|
| 1* | 5.9% |
| 2 | 3.3% |
| 3 | 5% |

*patient 1 was evaluated at 4 months

At 12 months, the following data was collected:

| Patient | % ratio of 11,11-D2-AA to AA |
|---|---|
| 1** | 10.5% |
| 2 | 6.9% |
| 3 | 8.6% |

**patient 1 was evaluated at 13 months

The above results evidence that a percent ratio of 11,11-D2-AA to AA in red blood cells of about 3% is necessary to achieve therapeutic results. This data further evidence that a percent ratio of 11,11-D2-AA to AA in red blood cells of about 5% is preferred; and that 6% or 8% is more preferred.

Regardless, the data as a whole show that the disease progression in a representative example of tauopathy is significantly reduced by the methods described herein.

What is claimed is:

1. A method for reducing lipid peroxidation in neurons, wherein said lipid peroxidation is associated with abnormal tubulin associated units (tau) characteristic of tauopathy, said method comprising:
contacting said neurons with a sufficient amount of a deuterated polyunsaturated fatty acid (PUFA) or ester or derivative thereof, over a period of time sufficient to accumulate said deuterated PUFA or ester or derivative thereof, in said neurons;
wherein said deuterated PUFA or ester or derivative thereof accumulated in said neurons stabilizes the neurons against neuronal death associated with abnormal tau.

2. A method for reducing lipid peroxidation in neurons of a patient, wherein said lipid peroxidation is associated with abnormal tubulin associated units (tau) characteristic of tauopathy, said method comprising:
administering to the patient a sufficient amount of a deuterated polyunsaturated fatty acid (PUFA) or ester or derivative thereof, over a period of time sufficient to accumulate said deuterated PUFA or ester or derivative thereof in the neurons inclusive of the neuronal membrane of said patient,
wherein said accumulated, deuterated PUFA or ester or derivative thereof attenuates lipid peroxidation in said neurons thereby stabilizing the neurons against neuronal death associated with abnormal tau.

3. A method of treating, ameliorating, or inhibiting the progression of a neurodegenerative disease or condition related to tauopathy in a subject, the method comprising: administering a first effective amount of one or more deuterated polyunsaturated lipids or pharmaceutically acceptable salts thereof to the subject during a first period of time.

4. The method of claim 3, wherein the neurodegenerative disease or condition related to tauopathy is selected from the group consisting of argyrophilic grain disease (AGD), chronic traumatic encephalopathy (CTE), corticobasal degeneration (CBD), frontotemporal dementia and parkinsonism, ganglioglioma, gangliocytoma, lipofuscinosis, lytico-bodigdisease, meningioangiomatosis, pantothenate kinase-associated neurodegeneration (PKAN), Pick's disease, postencephalitic parkinsonism, primary age-related tauopathy (PART), Steele-Richardson-Olszewski syndrome (SROS) (also referred to as progressive supranuclear palsy—PSP), subacute sclerosing panencephalitis (SSPE), Alzheimer's disease, or Lytico-bodig disease.

5. The method of claim 4, wherein the neurodegenerative disease or condition is suspected SROS.

6. The method of claim 1, wherein the deuterated PUFA or ester or derivative there of is selected from the group consisting of a deuterated fatty acid, a deuterated fatty acid ester, a deuterated fatty acid thioester, a deuterated fatty acid amide, a fatty acid deuterated phosphate ester, or a phospholipid derivative, and wherein at least one or more of the bis-allylic position position of the deuterated PUFA or ester or derivative thereof is a site of deuterium substitution.

7. The method of claim 6, further comprising deuterium substitution at at least one further allylic site.

8. The method of claim 6, wherein the polyunsaturated lipid has a structure of Formula (I): 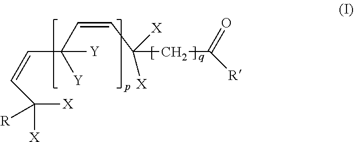
wherein:
R is hydrogen or optionally substituted C₁-C₁₀ alkyl, wherein said optional substitution is at least one deuterium;
R′ is —OR¹, —SR², —O(CH₂)CH(OR³)CH₂(OR⁴), —NR⁵R⁶ 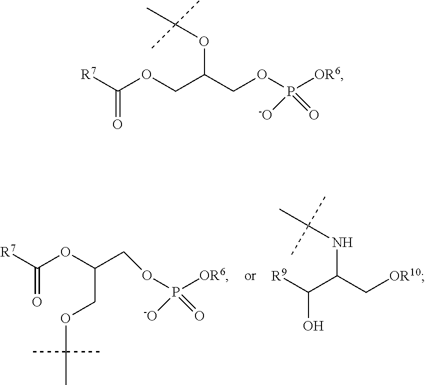
R¹ and R² is H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl;
each of R³ and R⁴ is independently H, optionally substituted —C(═O)C₁-C₂₁ alkyl, optionally substituted —C(═O)C₂-C₂₁ alkenyl, or optionally substituted —C(═O)C₂-C₂₁ alkynyl;
each of R⁵ and R⁶ is independently H, optionally substituted C₁-C₂₁ alkyl, optionally substituted C₂-C₂₁ alkenyl, optionally substituted C₂-C₂₁ alkynyl, optionally substituted C₃-C₁₀ cycloalkyl, optionally substituted C₆-C₁₀ aryl, optionally substituted 4 to 10 membered heteroaryl, or optionally substituted 3 to 10 membered heterocyclyl; or R⁵ and R⁶ together with the nitrogen atom to which they are attached form an optionally substituted 3 to 10 membered heterocyclyl;
R⁷ is optionally substituted C₁-C₂₁ alkyl, optionally substituted C₂-C₂₁ alkenyl, or optionally substituted C₂-C₂₁ alkynyl;
R⁹ is optionally substituted C₈-C₂₁ alkyl, optionally substituted C₈-C₂₁ alkenyl, or optionally substituted C₈-C₂₁ alkynyl;
R¹⁰ is H, 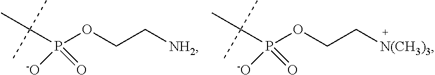
a mono-saccharide, a di-saccharide, or an oligosaccharide;
each X and Y is independently H or D, provided that at least one of X and optionally, one or more Y is D; and
each of p and q is independently an integer of 1, 2, 3, 4, 5, 6, 7, 8, 9 or 10;
or a mixture thereof.

9. The method of claim 8, wherein each of Y is D.

10. The method of claim 8, wherein R is methyl, C₄ alkyl, or C₇ alkyl, each optionally substituted with one or more D.

11. The method of claim 1, wherein the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

12. The method of claim 2, wherein the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

13. The method of claim 8, wherein the deuterated PUFA or ester or derivative thereof is deuterated linoleic acid, deuterated linolenic acid, deuterated arachidonic acid, deuterated eicosapentaenoic acid, deuterated docosahexaenoic acid, or a salt or an ester thereof.

14. The method of claim 8, wherein ester OR1 is an alkyl ester, a triglyceride, a diglyceride, or a monoglyceride.

15. The method of claim 14, wherein R1 is ethyl.

16. The method of claim 1, wherein the deuterated PUFA or ester or derivative thereof is selected from 11,11-D2-linoleic acid, 11,11,14,14-D4-linolenic acid, 13,13-D2-arachidonic acid, 7,7,10,10,13,13-D6-arachadonic acid, 7,7,10,10,13,13,16,16-D8-eicosapentaenoic acid, or 6,6,9,9,12,12,15,15,18,18-D10-docosahexaenoic acid, or ethyl esters thereof.

17. The method of claim 8, wherein the mixture of deuterated polyunsaturated lipids has a deuteration degree of at least 50% at the bis-allylic positions.

18. The method of claim 17, wherein the mixture of deuterated polyunsaturated lipids has a deuteration degree of at least 70% at the bis-allylic positions.

19. The method of claim 1, wherein the one or more deuterated PUFA or ester or derivative thereof are co-administered with at least one antioxidant.

20. The method of claim 2, wherein the one or more deuterated PUFA or ester or derivative thereof are co-administered with at least one antioxidant.

21. The method of claim 3, wherein the one or more deuterated PUFA or ester or derivative thereof are co-administered with at least one antioxidant.

22. The method of claim 21, wherein the antioxidant comprises Coenzyme Q, idebenone, mitoquinone, mitoquinol, vitamin C, or vitamin E, or combinations thereof.

23. The method of claim 4, wherein said frontotemporal dementia and parkinsonism is linked to chromosome 17 (FTDP 17).
